(19) United States
(12) Patent Application Publication
(10) Pub. No.: US 2026/0240483 A1
(43) Pub. Date: Aug. 20, 2026

(54) REMOVAL OF FAR-FIELD SIGNALS FROM ELECTROPHYSIOLOGY INFORMATION

(71) Applicant: CARDIOINSIGHT TECHNOLOGIES INC., INDEPENDENCE, OH (US)

(72) Inventors: Qingguo ZENG, Solon, OH (US); Qing LOU, Solon, OH (US); Timothy G. LASKE, Shoreview, MN (US)

(21) Appl. No.: 18/992,765

(22) PCT Filed: Jul. 17, 2023
(86) PCT No.: PCT/US2023/070323

§ 371 Date: Jan. 9, 2025

Related U.S. Application Data
(60) Provisional application No. 63/390,314, filed on Jul. 19, 2022.

(51) Int. Cl. A61B 5/367; A61B 5/00; A61B 5/343

(52) U.S. Cl. CPC A61B 5/367; A61B 5/343; A61B 5/6801

(57) ABSTRACT

In one example, a computer-implemented method includes computing first reconstructed electrophysiological signals on a cardiac envelope based on geometry data and electrophysiological data. The electrophysiological data represents electrophysiological signals measured non-invasively from locations distributed across a body surface, and the geometry data represents geometry for the cardiac envelope and geometry for the locations distributed on the body surface where the electrophysiological signals are measured. Second reconstructed electrophysiological signals are computed on the cardiac envelope based on the geometry data and the electrophysiological data, in which the second reconstructed electrophysiological signals being representative of far-field signal components. Near-field components of the electrophysiological signals are determined on at least a portion of the cardiac envelope based on the first and second reconstructed electrophysiological signals.

### CROSS-REFERENCE TO RELATED APPLICATION

This application claims the benefit of priority to U.S. Provisional Patent Application No. 63/390,314, filed Jul. 19, 2022, which is incorporated herein by reference in its entirety.

### FIELD

The present technology is generally related to removal of far-field signals from electrophysiology information.

### BACKGROUND

Electrophysiology involves measurements of voltage changes or electric current or manipulations such as associated with electrophysiological signals of the heart, brain or other anatomical structures. Electrophysiology studies are performed to measure and record electrophysiological signals from a patient's body, such as by placing one or more electrodes on and/or within the body. Some examples of EP studies that can be performed include electrocardiography, electroencephalography, electromyography, and the like. During these and other EP studies, there can be a variety of sources of interference, including far-field signals, which can affect signal measurements.

### SUMMARY

The techniques of this disclosure generally relate to determining far-field signal components from electrophysiology information. The techniques described herein can also be used to recover near-field components based on the far field components.

In one aspect, the present disclosure provides a computer-implemented method that includes computing first reconstructed electrophysiological signals on a cardiac envelope based on geometry data and electrophysiological data. The electrophysiological data represents electrophysiological signals measured non-invasively from locations distributed across a body surface, and the geometry data represents geometry for the cardiac envelope and geometry for the locations distributed on the body surface where the electrophysiological signals are measured. Second reconstructed electrophysiological signals are computed on the cardiac envelope based on the geometry data and the electrophysiological data, in which the second reconstructed electrophysiological signals being representative of far-field signal components. Near-field components of the electrophysiological signals are determined on at least a portion of the cardiac envelope based on the first and second reconstructed electrophysiological signals. In a further example, one or more non-transitory computer-readable media having instructions which, when executed by a processor, perform the method.

In another aspect, the disclosure provides a system that includes memory and at least one processor. The memory can store data and executable instructions, in which the data includes electrophysiological data and geometry data. The electrophysiological data represents electrophysiological signals measured from locations distributed across a body surface. The geometry data represents geometry for a surface of interest and geometry for the locations distributed on the body surface where the electrophysiological signals are measured. The processor is configured to access the memory and execute the instructions to at least:
compute first reconstructed electrophysiological signals on the surface of interest based on the geometry data and the electrophysiological data;
compute second reconstructed electrophysiological signals on the surface of interest based on the geometry data and the electrophysiological data, the second reconstructed electrophysiological signals being representative of far-field signal components; and
determine near-field components for the electrophysiological signals for at least a portion of the surface of interest based on the first and second reconstructed electrophysiological signals for the portion of the surface of interest.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram that illustrates an example system to generate near-field electrophysiological data.

FIG. 2 is a block diagram of a source node placement function.

FIGS. 3A and 3B are conceptual diagrams showing part of an example method for reconstructing electrophysiological signals.

FIGS. 4A and 4B are conceptual diagrams showing part of another example method for reconstructing electrophysiological signals.

FIG. 5 is a block diagram that illustrates an example reconstruction engine.

FIG. 6 is a block diagram that illustrates an example system for mapping and analysis of electrophysiological signals.

FIG. 7 is a flow diagram of a method to determine near-field electrophysiological data.

FIGS. 8-17 depict example graphical outputs and associated signals, such as can be generated by the system of FIG. 6 or method of FIG. 7.

### DETAILED DESCRIPTION

This description relates to systems and methods to provide a measure of near-field electrophysiological signals. As described herein, this can be implemented by removing contributions of respective far-field signals from electrophysiological signals of interest.

As described herein, systems and methods described herein can be implemented as machine-readable instructions executable by a processor. The processor includes code (e.g., a reconstruction engine) programmed to compute first reconstructed electrophysiological signals on a cardiac envelope or other surface of interest (e.g., a cardiac surface or a virtual surface) based on geometry data and electrophysiological data. For example, reconstruction engine uses a method of fundamental solutions (MFS) to solve the inverse problem for reconstructing the electrophysiological signals on the cardiac envelope, such as disclosed in U.S. Pat. No. 7,983,743, which is incorporated herein by reference in its entirety. The electrophysiological data represents electrophysiological signals measured (e.g., unipolar signal measured by an arrangement of body surface sensors) non-invasively from locations distributed across a body surface. The geometry data represents spatial geometry for the cardiac envelope and spatial geometry for the locations distributed on the body surface where the electrophysiological signals are measured (e.g., sensor locations). For example, the geometry data can represent respective locations (e.g., as absolute or relative spatial coordinates) in a common three dimensional spatial coordinate system.

The reconstruction engine is also programmed to compute second reconstructed electrophysiological signals on the cardiac envelope based on the geometry data and the electrophysiological data. However, the reconstruction engine is programmed (e.g., using MFS) to compute the second reconstructed electrophysiological signals based on locations of virtual source nodes that are different than used to compute the first reconstructed electrophysiological signals. For example, virtual source nodes outside the body can be moved further (away) from a geometric center of body. Additionally, or alternatively, virtual source nodes within the heart can be moved toward (closer to) a geometric center of heart. Thus, the second reconstructed electrophysiological signals are representative of (or emphasize) far-field signal components. As used herein, far-field signals or signal components refer to signals originating far away (e.g., a distance greater than a threshold distance) from a measurement location and/or a location (node) where a signal is reconstructed on a cardiac envelope. In some examples, the locations of the source nodes can be adaptively determined relative to the respective locations on the cardiac envelope and/or the body surface. The processor also includes code (e.g., a near-field calculator) programmed to determine near-field components of the electrophysiological signals on at least a portion of the cardiac envelope based on a difference between the first and second reconstructed electrophysiological signals. Systems and methods described herein can use the near-field components of the electrophysiological signals reconstructed on at least a portion of the cardiac envelope to control delivery of a therapy to

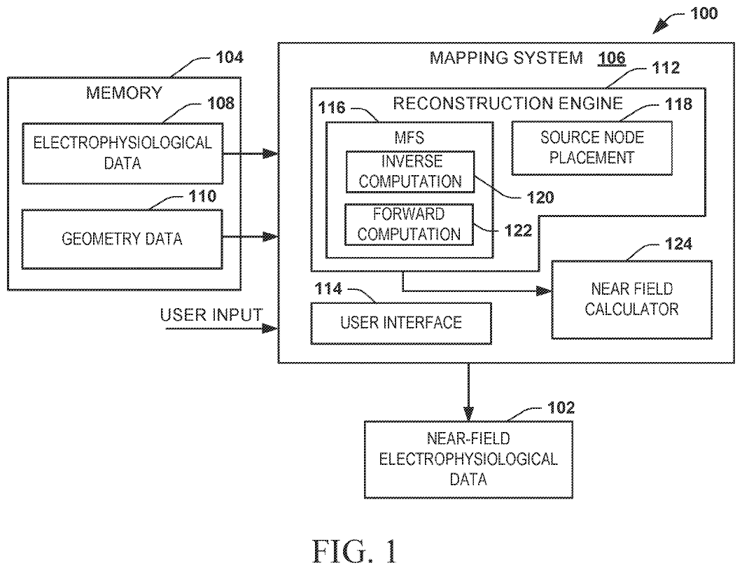

FIG. 1 depicts an example system 100 to generate near-field electrophysiological data 102 representative of electrophysiological signals reconstructed onto one or more surface of interest. In some examples, the surface of interest is a cardiac envelope. As used herein, a cardiac envelope may refer to any two-dimensional or three-dimensional surface or surfaces residing inside the patient's body on to which electrical signals are to be reconstructed. As one example, the surface corresponds to a virtual surface (e.g., a sphere or other three-dimensional structure). As another example, the surface corresponds to one or more surfaces of an anatomical structure, such as an epicardial surface, an endocardial surface or both epicardial and endocardial surfaces. The cardiac envelope thus may be configured as a cardiac surface model having three-dimensional geometry that is registered in or can be registered into a spatial coordinate system of a patient's anatomy. The cardiac surface model may include a cardiac nodes distributed across the geometry representing the cardiac envelope.

As an example, the system 100 can be implemented as a computing apparatus that includes memory 104 and a processor configured to execute instructions, shown in FIG. 1 as a mapping system 106. The memory 104 can be implemented as one or more non-transitory machine-readable media configured to store data and instructions. The processor is configured to access the memory and execute the instructions to perform the methods and functions corresponding to the mapping system 106. The memory 104 stores electrophysiological data 108, such as representing unipolar electrophysiological signals measured by an arrangement of electrodes (e.g., distributed across the body surface and/or invasive electrodes) over one or more time intervals. The electrophysiological data 108 may include real time measurements and/or previous measurements, which generally may vary depending on whether the system 100 is being utilized for real time analysis (e.g., during an electrophysiological study) or post-procedure analysis.

The memory 104 also stores geometry data 110. The geometry data 110 includes data representing body surface geometry for the locations distributed on the body surface where the electrophysiological signals are measured. For example, the locations on the body surface correspond to respective electrode locations of a sensing system (e.g., an arrangement of sensors) that is positioned on the patient's thorax and configured to sense body surface electrophysiological signals from such electrode locations. Examples of a non-invasive sensing system that can be employed to measure body surface electrophysiological signals are shown and described in U.S. Pat. No. 9,655,561 and International publication No. WO 2010/054352, each of which is incorporated herein by reference.

The geometry data 110 includes data representing geometry of a surface of interest, such as a cardiac envelope for which the reconstructed electrophysiological signals are determined. The cardiac envelope can correspond to a three dimensional epicardial surface geometry of a heart. Alternatively or additionally, the cardiac envelope can correspond to a three dimensional endocardial surface geometry of the heart. As yet another alternative, the cardiac envelope may correspond to virtually any geometric surface that resides between a region inside the patient's heart and the outer surface of the patient's torso where the electrical measurements are taken. The geometry data 110 may correspond to actual patient anatomical geometry, a preprogrammed generic model or a combination thereof (e.g., a model that is modified based on patient anatomy).

As an example, the geometry data 110 may be derived from processing image data acquired for the patient via an imaging system (not shown). For example, the imaging system can be implemented according to any imaging modality, such as computed tomography (CT), magnetic resonance imaging (MRI), x-ray, fluoroscopy, ultrasound or the like, to acquire three-dimensional image data for the patient's torso. Such image processing can include extraction and segmentation of anatomical features, including one or more organs and other structures, from a digital image set. Additionally, a location for each of the electrodes in the sensing system can be included in the geometry data 110, such as by acquiring the image while the electrodes are disposed on the patient and identifying the electrode locations in a coordinate system through appropriate extraction and segmentation. The imaging may be performed concurrently with recording the electrophysiological signals that is utilized to generate the patient measurement data 170 or the imaging can be performed separately (e.g., before or after the measurement data has been acquired). In another example, one or more non-imaging based techniques can also be utilized to provide a three-dimensional position of the electrodes in the coordinate system, such as a digitizer or manual measurements.

In the example of FIG. 1, the mapping system 106 includes a reconstruction engine 112. The reconstruction engine 112 is programmed to reconstruct electrophysiological signals on to the cardiac envelope based on the electrophysiological data 108 and the geometry data 110. As described herein, reconstruction engine 112 is programmed to compute first and second sets of reconstructed electrophysiological signals for a surface of interest, such as a cardiac envelope. For example, the reconstruction engine 112 is configured to compute each of the respective sets reconstructed electrophysiological signals for a plurality of cardiac nodes spatially distributed over the cardiac envelope based on the same electrophysiological data 108 measured non-invasively over one or more time intervals. In some examples, the number of cardiac nodes can be greater than 1,000 or 2,000 or more depending upon a desired resolution. For example, the surface region or regions that define the cardiac envelope and/or time interval(s) for which the reconstructed electrophysiological signals are computed may be set and stored in the memory 104 in response a user input instruction entered through a user interface 114.

In the example of FIG. 1, the reconstruction engine 112 includes code programmed to implement an MFS 116 and a source node placement function 118. The MFS 116 includes an inverse computation 120 and a forward computation 122, which solve the inverse problem to generate respective first and second electrophysiological signals on the surface of interest based on the electrophysiological data 108, the geometry data 110 and source node data provided by the source node placement function 118. For example, the geometry data 110 defines spatial coordinates of nodes on the surface of interest (e.g., cardiac nodes on a spatial envelope) for which the reconstructed electrophysiological signals are to apply and spatial coordinates of nodes on the body surface (e.g., body surface nodes) that define the electrode locations where electrophysiological signals are measured from the body surface to provide the electrophysiological data 108. The spatial coordinates of the respective nodes can be provided in a three-dimensional coordinate system that is registered with patient anatomy.

The source node placement function 114 is programmed to provide the source node data representative of a plurality of source nodes (e.g., virtual nodes or fictitious points) in the three-dimensional coordinate system of the geometry data 110. The source nodes can include body surface source node and cardiac source nodes. The body surface source nodes can represent virtual nodes at locations spaced radially outwardly from the outer surface of the patient's body where the electrophysiological signal measurements are made. The cardiac source nodes can represent virtual nodes spaced radially inwardly from the cardiac surface (or other surface of interest). As described herein, the source nodes used by the MFS 116 to compute the second reconstructed electrophysiological signals are different from the source nodes used by the MFS to compute the first reconstructed electrophysiological signals. By using different source nodes in this way, one of the first or second electrophysiological signals are smoothed as to be more representative of (e.g., it emphasizes) far-field signal components than the other. In the following examples, the second reconstructed electrophysiological signals are described as being representative of the far-field signals.

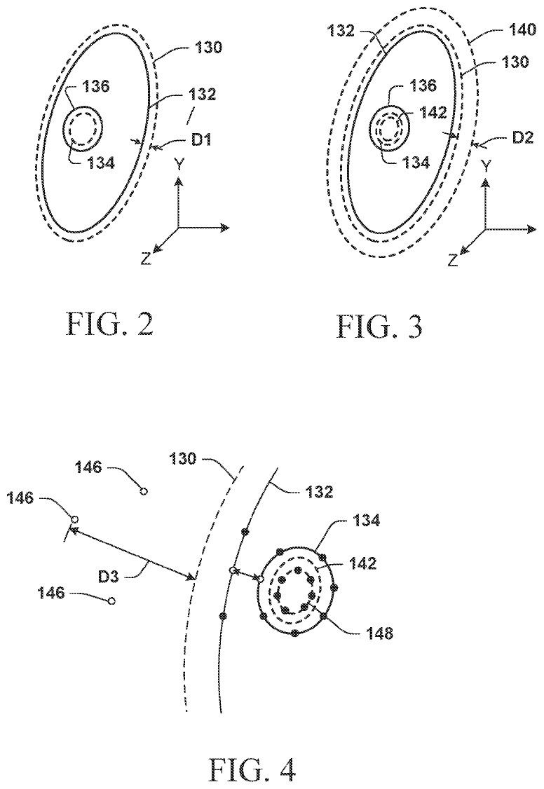

As an example, the source node placement function 114 is programmed to provide first source node data, which describes first source node locations to be used (e.g., by MFS 116) for determining the first set of reconstructed electrophysiological signals. The source node placement function 114 can provide the first source node locations at default or fixed spatial distances relative to the body surface nodes and cardiac nodes. For example, with reference to FIG. 2, first body source nodes can be placed at locations along a virtual first surface 130 spaced a predetermined distance radially outwardly from the body surface 132 on which the body surface nodes reside. Similarly, the first cardiac source nodes can be placed at locations along a surface 134 spaced a predetermined distance radially inwardly from the cardiac envelope, shown conceptually as 136, onto which the EP signals are being reconstructed. In an example, the source node placement function 118 includes a distance calculator programmed to compute the distance from the respective surface nodes (e.g., body surface or cardiac surface nodes) to respective locations for the source nodes, such as the distance along a normal line drawn with respect to a tangent line at the respective surface nodes.

The source node placement function 118 (e.g., the distance calculator thereof) is also programmed to determine locations of respective second body source nodes and second cardiac source nodes for use (e.g., by the MFS 116) in determining the second set of reconstructed electrophysiological signals. The respective second body source nodes and second cardiac source nodes can be placed uniformly or adaptively, such as based on the locations of first source nodes.

In an example, with reference to FIG. 3, each of the second body source nodes can be placed on a surface 140 spaced radially outwardly a predetermined (e.g., uniform) distance, shown as D2, from respective locations of the first body source nodes (e.g., on virtual surface 130). The predetermined distance for the second body source nodes can be calculated by applying a multiplier greater than one (e.g., 1.5×, 2×, 3×, 5× or more) to distance D1 between respective first body source nodes and the body surface 132. Each of the second cardiac source nodes can likewise be placed on a surface 142 spaced radially inwardly a predetermined (e.g., uniform) distance from respective locations of the first cardiac source nodes (e.g., on surface 134). For example, the distance for placing the second cardiac source nodes radially inward from the cardiac surface can be calculated by applying a multiplier that is less than one (e.g., 0.9×, 0.8×, 0.7×, 0.5 or less) to the distance between respective first cardiac source nodes and the geometric center of the heart. Other multipliers or distance computations can be used to place the respective second body and cardiac source nodes uniformly relative to the body and cardiac surfaces.

In another example, the source node placement function 118 is programmed to adaptively determine locations for the respective second body source nodes and second cardiac source nodes. The adaptive source node placement function can set the distance of the respective source nodes according to a predefined function and/or in response to a user input entered through the user interface. The source node placement function 118 can determine locations for selected ones of the second source nodes to adjust (e.g., increase) smoothness of the far-field signal components in the second set of reconstructed electrophysiological signals.

For example, with reference to FIG. 4, the source node placement function 118 can place each of the second body source nodes 146 at a respective location that is spaced a distance D3 outwardly from the first surface 130 wherein the first body source nodes were placed. Alternatively, the distance can be determined from the body surface 132. The distance D3 can be variable for each of the second body source nodes 146. In an example, the distance D3 between the first surface 130 and a given second body source node 146 depends on (e.g., is inversely proportional to) the distance between one or more associated cardiac nodes on cardiac envelope 134 and the nearest electrode location(s) on the body surface 132. Thus, second body source nodes 146, which are associated with cardiac nodes on envelope 134 that are closer to the body surface electrodes, can be moved further away from the body surface 132 than other second body source nodes associated with cardiac nodes on the envelope 134 that spaced further from respective electrodes on the body surface. As a result of such adaptive placement of second body source nodes, such as shown in FIG. 4, the reconstructed electrophysiological signals computed by the MFS 116 using the second body source nodes can exhibit increased smoothing of far-field signal components compared to the uniform placement of second body source nodes, such as shown in FIG. 3.

As a further example, the source node placement function 118 is programmed to determine a closest distance between the respective cardiac nodes on the cardiac envelope 134 and electrode locations (nodes) on the body surface 132. The source node placement function 118 can then identify a set of one or more cardiac nodes for which the distance is less than a distance threshold. The distance threshold can be a default value or variable, such as responsive to a user input via the user interface 114. The source node placement function 118 can then place the second body surface source nodes, which are associated with the identified cardiac nodes, to locations spaced from the body surface a distance that is inversely proportional to the determined distance. Other second body surface source nodes, which are not associated with the identified cardiac nodes, can be placed (e.g., remain) at their respective first body surface source node locations. Alternatively, such other second body surface source nodes can be placed uniformly a distance outwardly from the body surface or outwardly from the respective first body surface source node locations, such as described herein. This results in the corresponding second body surface node locations being spaced further from the body surface than their respective first body surface source node locations.

Also, as shown in FIG. 4, the source node placement function 118 is programmed to place the second cardiac source node at locations radially inwardly from the cardiac envelope. The second cardiac source node locations can be uniformly or adaptively placed. In one example, the source node placement function 118 is programmed to place the second cardiac source nodes at locations along a virtual surface 148 spaced a predetermined (e.g., uniform) distance radially inwardly from the cardiac envelope 134 onto which the EP signals are being reconstructed. The surface 148 can also be radially inward from the surface 142 where the first cardiac source nodes are placed. In another example, the source node placement function 118 is programmed to place the second cardiac source nodes at locations along the same surface 142 where the first cardiac source nodes are placed (e.g., the first and second cardiac source nodes can be the same). In yet another example, the source node placement function 118 is programmed to place the second cardiac source nodes at locations adaptively determined based on the distance between the cardiac envelope 134 and nearest electrodes on the body surface 132.

Referring back to FIG. 1, the MFS 116 can be programmed to use the source node data for both the inverse and forward computations 120 and 122. For example, the MFS 116 is further programmed (e.g., to include or otherwise utilize a matrix calculator) to derive an analytical expression for the method of fundamental solutions that includes a transfer matrix A (also referred to herein as the A matrix). As disclosed herein, the A matrix includes coefficients that relate a location of each source node (e.g., including both cardiac source nodes and body surface source nodes) to the body surface node locations distributed on the body surface where the electrophysiological signals are measured. The inverse computation 120 is programmed to perform an inverse computation on the A matrix and the noninvasively measured electrophysiological signals provided by the electrophysiological data 108 to compute a plurality of source node coefficients.

The MFS 116 is also programmed (e.g., to include or otherwise utilize another matrix calculator) to determine a matrix of coefficients B (also referred to as the B matrix) that relates each cardiac node location on the cardiac envelope to each source node location. The forward computation 122 is programmed to perform a forward computation based on the B matrix and the plurality of source node coefficients to compute the cardiac electrophysiological signals on the cardiac envelope. The reconstruction engine 112 may thus compute the first and second reconstructed electrophysiological signals on the cardiac envelope for each of a plurality of consecutive time samples in one or more time intervals. As described herein, the reconstruction engine 112 employs the MFS using the first source nodes (e.g., source node data provided by source node placement function 118 to describe first body source nodes and first cardiac source nodes) to compute the first reconstructed electrophysiological signals based on the EP data 108 and the geometry data 110. The reconstruction engine 112 also employs the MFS using the second source nodes (e.g., source node data provided by source node placement function 118 to describe second body source nodes and second first cardiac source nodes) to compute the second reconstructed electrophysiological signals based on the EP data 108 and the geometry data 110. As described herein, the first and second reconstructed electrophysiological signals are computed on the same cardiac envelope based on the same set of electrophysiological and geometry data 108 and 110.

In another example, the reconstruction engine 112 is programmed to compute the second reconstructed electrophysiological signals on the surface of interest (e.g., the cardiac envelope) based on the first reconstructed electrophysiological signals. For example, the reconstruction engine 112 can compute an average value (or other smoothing function) for the first reconstructed electrophysiological signals in a spatial neighborhood of respective cardiac nodes on the cardiac envelope. In this way, the second reconstructed electrophysiological signals represent smoothed signals across the cardiac envelope within a spatial distance of the respective cardiac nodes. The size of the neighborhood can be set as a number of nodes or a spatial distance across the cardiac envelope. The neighborhood size can be set in response to a user input instruction entered through the user interface 114 (e.g., a knob, button or slide graphical user interface) to control an amount of smoothing being implemented for generating the second reconstructed electrophysiological signals.

As a further example, the reconstruction engine 112 can be programmed to implement normalized weighted averaging. For example, the weighted averaging can be implemented using Gaussian convolution, which assigns higher weights to nearby nodes than further nodes, and where the sum of weights add up to be 1. The application of the weighting function further can be controlled based on a spatial distance (e.g., a distance<3 cm) or based on neighborhood layers and sigma values that can be set for the Gaussian kernel.

The mapping system 106 also includes a near-field calculator 124 configured to provide the near-field electrophysiological data 102 based on the first and second reconstructed electrophysiological signals. In an example, the MFS computes the first and second reconstructed electrophysiological signals on the same cardiac envelope based on the same set of electrophysiological and geometry data 108 and 110. electrophysiological signals due to far-field smoothing. Thus, by subtracting the second reconstructed electrophysiological signals from the first reconstructed electrophysiological signals, far-field signal components can be reduced or even removed so the resulting near-field electrophysiological data 102 are more representative of near-field signal components. The near-field electrophysiological data 102 can be determined for the entire cardiac envelope (e.g., a heart surface) or for a selected region of the cardiac envelope. For example, a user provides a user input through the user interface 114 to select one or more signal intervals and/or specify a region of interest on the heart. The reconstruction engine 112 can compute the compute near-field electrophysiological data 102 responsive to the user input. Corresponding output data can in turn be generated and rendered as a corresponding graphical output for display on an output device, such as described herein.

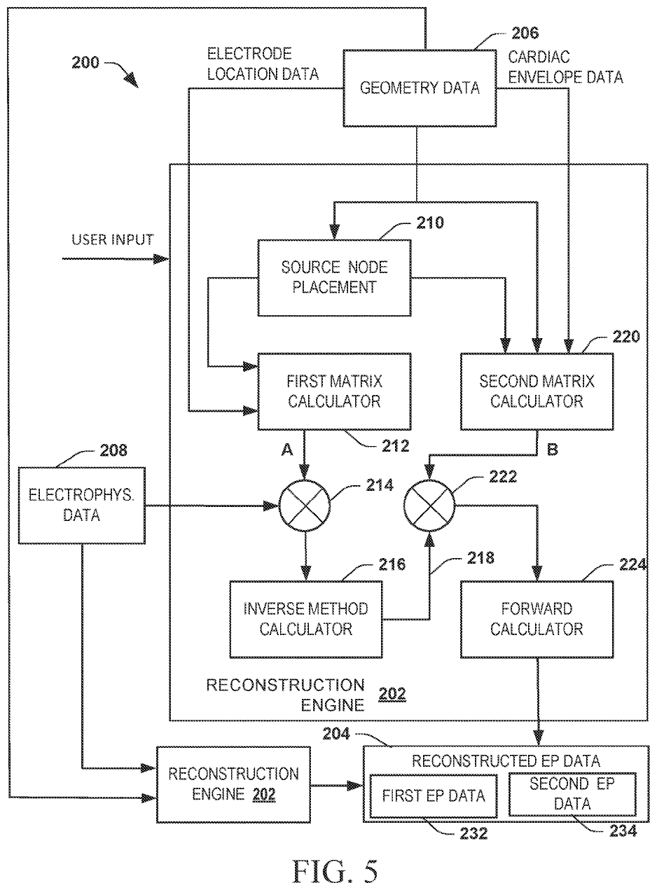

FIG. 5 depicts an example of a system 200 configured to reconstruct electrophysiological signals on a cardiac envelope. The system 200 includes a reconstruction engine 202, such as corresponding to the reconstruction engine 112 of FIG. 1, which is demonstrated as a workflow diagram of program code elements that may be executed by one or more processors. There can be multiple (e.g., two or more) instances of the reconstruction engine 202, which operate in parallel. For example, each instance of reconstruction engine 202 is programmed to compute respective sets of reconstructed EP data 204 representative of electrophysiological signals reconstructed on a surface of interest. The near-field calculator can thus determine near-field components of the electrophysiological signals based on combining respective sets of the reconstructed EP data, as described herein.

In the example of FIG. 5, each instance of reconstruction engine 202 implements MFS (e.g., an example of MFS 116) to compute reconstructed electrophysiological signals data 204 based on geometry data 206 and electrophysiological data 208 (e.g., corresponding to EP data 108 and geometry data 110). The geometry data 206 is generated to specify the geometrical relationship between electrode locations and the cardiac envelope onto which the electrophysiological signals are being reconstructed. Additionally, the electrophysiological data 208 can represent unipolar EP signals measured by each electrode in an arrangement of electrodes (non-invasively at body surface node locations represented the in geometry data). In some examples, the electrophysiological data 208 can also include EP signals measured by one or more electrodes positioned within the body (e.g., invasive EP measurements).

As a further example, the reconstruction engine 202 includes a source node placement function 210 configured to determine the locations of first and second source nodes, which are virtual (e.g., fictitious points) used by the MFS implemented by the reconstruction engine 202. As disclosed herein, the source nodes include the first and second sets of respective source nodes, each of which includes body source nodes and cardiac source nodes. In some examples, the source node placement function 210 places the first body source nodes in a spatial arrangement and distribution positioned radially outwardly from the spatial distribution of electrode locations on the body surface where the electrophysiological signals are measured. The second body source nodes can be positioned in a three-dimensional spatial arrangement and distribution radially outwardly of the spatial arrangement and distribution of the first body source nodes. As described herein, the source node placement function 210 can position the body source nodes uniformly or adaptively in three-dimensional space, such as by controlling the distance of such nodes relative to the body surface, the cardiac envelope or another anatomical surface or virtual location.

Additionally, the source node placement function 210 places the first cardiac source nodes in a three-dimensional spatial arrangement and distribution positioned radially inwardly of the spatial arrangement and distribution of the cardiac nodes on the cardiac envelope. The second cardiac source nodes can be placed in a three-dimensional spatial arrangement and distribution positioned at the same locations or radially inwardly of the first cardiac source nodes. In some examples, the first and second source nodes can be implemented with the same number of nodes or with a different number of nodes than the respective body nodes (e.g., electrode locations) and cardiac node on the cardiac envelope. For example, each set of first and second source nodes may have a greater or lesser number of nodes than the respective cardiac and body surface nodes. The number of nodes and their spatial distribution can be set to a default or user-programmable value (e.g., responsive to a user input).

The reconstruction engine 202 also includes a first matrix calculator 212 programmed to compute a transfer matrix A that relates the location of each source node (e.g., determined by source node placement function 210) to the geometry of the body surface nodes, which correspond to locations distributed on the body surface where the electrophysiological signals are measured. The first matrix calculator 212 thus computes a respective transfer matrix A for the first and second sets of source nodes. The coefficients in the transfer matrix A are representative of the “strength” of each source node. For example, the measured electrophysiological signals on the body surface may be expressed as a vector (V_{BS}):

$${V}_{BS} = A Γ$$

where the transfer matrix A is a 2N×P+1 matrix,
   in which: N represents the total number of body surface nodes, and
      P represents the total number of source nodes.

In this example, the first matrix calculator 212 may be configured to compute the value of each entry (a_{j,k}) in the matrix A as a function of the distance between each body surface node and each source node. For example, the value of each entry a_{j,k} in the matrix A is a function of the distance between body surface (e.g., torso) node (TNⱼ) and source node (SNₖ) in the spatial coordinate system, such that:

$${a}_{j , k} = \frac{1}{{r}_{j , k}}$$

where r_{j,k} equals the distance between a body surface node TNⱼ and source node SNₖ in the space.

For example, the distance between each body surface node and each of the source nodes (e.g., each of the body surface source nodes and cardiac source nodes) may be computed between the respective locations of such nodes according to a Euclidean or other distance calculation. Because each value for r_{j,k} is readily calculable in view of the known coordinates of each torso node and each source node, the entries a_{j,k} in matrix A are likewise known.

A combinatorial function 214 of the reconstruction engine 202 thus can employ the computed transfer matrix A to express the non-invasively measured electrophysiological data 208 as a function of the transfer matrix A and Γ, such as described above. Therefore, the 1×P+1 vector Γ is the only unknown in this expression. An inverse method calculator 216 is programmed to perform an inverse computation on the A matrix and the noninvasively measured electrophysiological signals to compute a plurality of source node coefficients. In this way the inverse method calculator 216 determines the value of the inverse of the transfer matrix (e.g., Γ=A⁻¹*V_{BS}). Since the computation of Γ is an ill-posed problem, the inverse method calculator 216 can employ any of a variety of mathematical schemes to estimate the values in the matrix Γ. Examples of schemes that are believed to provide effective results for computing Γ include Tikhonov zero order regularization and the Generalized Minimal Residual (GMRes) method. For example, the inverse method calculator 216 can be programmed to implement Tikhonov regularization, such as described in U.S. Pat. No. 6,772,004, or GMRes regularization, such as described in U.S. Pat. No. 7,016,719, each of which is incorporated herein by reference in its entirety.

The reconstruction engine 202 also includes a second matrix calculator 220 to compute a matrix B. The matrix B operates to translate the source node coefficients determined via the inverse method calculator 216 to corresponding electrophysiological signals on the cardiac envelope of interest at each cardiac node location (e.g., endocardial nodes and/or epicardial nodes). As an example, the value of each entry b_{j,k} in matrix B is a function of the distance between each cardiac node CNⱼ and source node SNₖ, such that:

$${b}_{j , k} = \frac{1}{{r}_{j , k}}$$

where r_{j,k} equals the distance between cardiac node CNⱼ and source node SNₖ.

In the example of FIG. 5, another combinatorial function 222 is configured to express the cardiac electrophysiological signals as a function of the transfer matrix B and T, such as determined above by the inverse method calculator 216. For example, the combinatorial function 222 can express the cardiac electrophysiological signals on the cardiac envelope (V_{CE}) as a function of the matrix B and Γ, such as:

$${V}_{CE} = B Γ$$

where B is a M×P+1 matrix,
   where M represents the total number of cardiac nodes and
      P represents the total number of source nodes (e.g., cardiac
   and body source nodes).

As the distance for each value for r_{j,k} is readily calculable, the entries in matrix B are likewise known, which allows for a straightforward calculation of V_{CE} from B and Γ. For example, a forward calculator 224 is configured to compute the corresponding estimate of reconstructed electrophysiological data 204 on the cardiac nodes distributed across the cardiac envelope. In some examples, the locations of the plurality of cardiac nodes are set, in response to a user input, such as to reside on a selected one or both of an epicardial surface and an endocardial surface or another cardiac envelope. The reconstruction engines 202 are thus configured to compute the reconstructed electrophysiological data 204 to include first electrophysiological data 230 and second electrophysiological data 232. For example, one instance of the reconstruction engine 202 computes the first electrophysiological data 230 based on the electrophysiological data 208 and the geometry data 206 and according to a first source nodes (e.g., determined by the source node placement function 210). Another instance of the reconstruction engine 202 computes the second electrophysiological data 232 based on the electrophysiological data 208 and the geometry data 206 and according to the second set of source nodes (e.g., determined by the source node placement function 210). The first and second electrophysiological data 230 and 232 can be stored in memory for further processing (e.g., to determine near-field electrophysiological signals), as described herein.

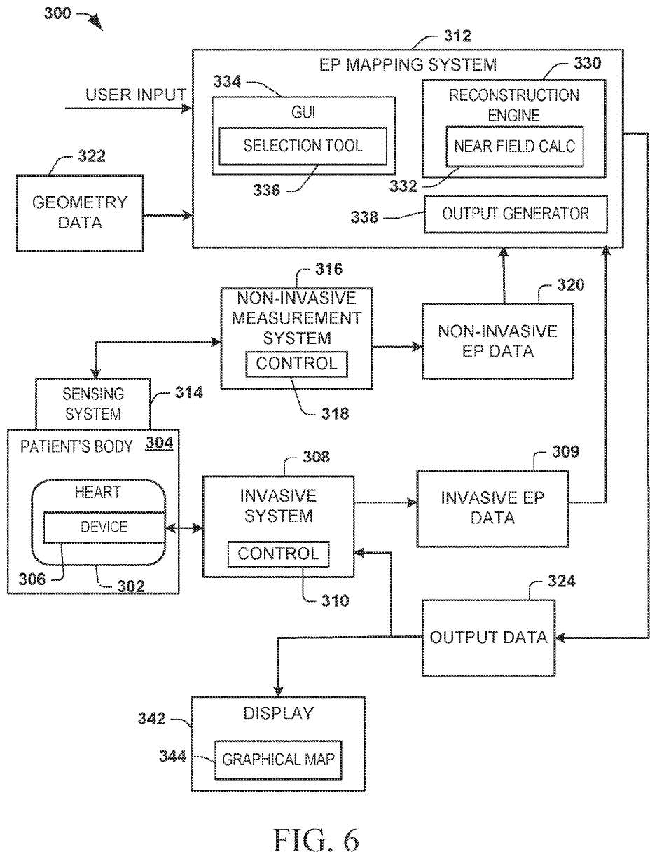

FIG. 6 depicts an example of a system 300 that can be utilized for performing diagnostics and/or treatment of a patient. In some examples, the system 300 can be implemented to generate corresponding graphical outputs for signals and/or graphical maps for a patient's heart 302 in real time as part of a diagnostic procedure (e.g., monitoring of signals during an electrophysiology study) to help assess the electrophysiological signals for the patient's heart. Additionally or alternatively, the system 300 can be utilized as part of a treatment procedure, such as to provide and/or help a physician determine one or more parameters for delivering a therapy (e.g., delivery location, amount and/or type of therapy) and provide a visualization and/or other output to control and/or facilitate determining when to end the delivery of the treatment.

For example, an invasive device 306, such as a catheter or other probe, can be inserted into a patient's body 304. The invasive device 306 can include one or more electrodes affixed thereto to deliver a treatment (e.g., via contact or not contact) to the patient's heart 302, endocardially or epicardially. Those skilled in the art will understand and appreciate various types and configurations of devices 306, which can vary depending on the type of treatment and the procedure. The placement of the device 306 can be guided via a localization or tracking system (not shown), which can operate to localize the device 306 in a 3D coordinate system.

The device can be implemented as part of an invasive system 308. The invasive system 308 can include a control 310 configured to process (electrically) and control the capture of the measured signals as to provide corresponding invasive EP measurement data 309. The control 310 can also be configured to control the delivery of therapy by the device 306, such as based on the near-field components of electrophysiological signals estimated for at least a portion of the cardiac envelope.

In an example, the device 306 can include one or more electrodes disposed thereon at predetermined locations with respect to the device. Each such electrode can be configured to deliver an electrical signal, which can be localized. The device 306 can provide the signal as to deliver a localization specific therapy, such as ablation, a pacing signal or to deliver another therapy (e.g., providing electrical therapy, or controlling delivery of chemical therapy, sound wave therapy, or any combination thereof). For instance, the device 306 can include one or more electrodes located at a tip of a pacing catheter, such as for pacing the heart, in response to electrical signals (e.g., pacing pulses) supplied by the system 308. Other types of therapy can also be delivered via the system 308 and the device 306 that is positioned within the body 304. The therapy delivery means can be on the same catheter or a different catheter probe than is used for sensing electrophysiological signals invasively.

As a further example, the system 308 can be located external to the patient's body 304 and be configured to control therapy that is being delivered by the device 306, such as based on the output data 324. For instance, the system 308 can also control electrical signals provided via a conductive link electrically connected between the delivery device (e.g., one or more electrodes) 306 and the system 308. The control system 310 can control parameters of the signals supplied to the device 306 (e.g., current, voltage, repetition rate, trigger delay, sensing trigger amplitude) for delivering therapy (e.g., ablation or stimulation) via the electrode(s) on the invasive device 306 to one or more location on or inside the heart 302. The control can be based on output data 324, which provided according to near-field components of electrophysiological signals determined for at least a portion of the cardiac envelope. The control circuitry 310 can set the therapy parameters and apply stimulation or other therapy based on automatic, manual (e.g., user input) or a combination of automatic and manual (e.g., semiautomatic) controls. One or more sensors (not shown but could be part of the device) can also communicate sensor information back to the control 310. In some examples, the invasive system 308 and device 306 can be omitted from the system 300.

A sensing system 314 includes one or more sensors configured to measure electrophysiological signals non-invasively from the patient's body 304. As one example, the sensing system 314 can correspond to a high-density arrangement of body surface sensors that are distributed over a portion of the patient's outer body surface (e.g., thorax) for measuring electrophysiological signals associated with the patient's heart (e.g., as part of an electrocardiographic mapping procedure). Examples of non-invasive sensors that can be used to implement the sensing system 314 are shown and described in U.S. Pat. No. 9,655,561 International patent publication no. WO2010054352A1, each of which is incorporated herein by reference. Other arrangements and numbers of sensors can be used as the sensing system 314. As an example, the sensors can be configured as a sheet or patch, which does not cover the patient's entire torso and is designed for measuring electrophysiological signals for a particular purpose (e.g., an arrangement of electrodes specially designed for analyzing a selected type of arrhythmia) and/or for monitoring electrophysiological signals at a predetermined spatial region of the heart.

The electrophysiological signals (e.g., potentials) measured non-invasively via the sensing system 314 are provided to the measurement system 316. The measurement system 316 can include appropriate controls and signal processing circuitry 318 for providing corresponding EP measurement data 320 that describes electrophysiological signals measured by the electrodes in the sensing system 314. The measurement data 320 can include analog and/or digital information (e.g., corresponding to electrophysiological data 108).

The non-invasive measurement control 318 can also be configured to control the data acquisition process (e.g., sample rate, line filtering, baseline filter etc.) for measuring electrophysiological signals and providing the non-invasive EP data 320. In some examples, the control 318 can control acquisition of measurement data 320 separately from the therapy system operation, such as in response to a user input. In other examples, the measurement data 320 can be acquired concurrently with and in synchronization with delivering therapy using the device 306, such as to detect electrophysiological signals of the heart 302 responsive to applying a given therapy (e.g., according to therapy parameters).

An EP mapping system 312 includes an electrogram reconstruction engine 330 (e.g., corresponding to reconstruction engine 112, 202), which is programmed to reconstruct electrophysiological signals on a cardiac envelope, such as disclosed herein. For example, reconstruction engine 330 includes an MFS programmed to perform inverse and forward computations to electrophysiological signals reconstructed on a cardiac envelope based on geometry data 322 and the EP data 320. As described herein, the reconstruction engine 330 can implement the MFS based on geometry data 322 and the EP data 320 to derive first and second sets of the reconstructed electrophysiological signals using different source node locations. A near-field calculator 332 is programmed to determine electrophysiological signals representative of near-field signals based on a difference between the first and second sets of the reconstructed electrophysiological signals. That is, by determining the second set of reconstructed electrophysiological signals to be representative of far-field electrophysiological signals, the calculator 332 can subtract such signals from the first set to describe near-field electrophysiological signals on the cardiac envelope. The cardiac envelope where the signals are reconstructed can describe an entire 3D cardiac surface or a region or interest, such as can be selected in response to a user input (via GUI 334). In an example, the GUI can include a selection tool 336 through which a user can select one or more signal intervals of interest (e.g., one or more beats) in response to a user input. Additionally, or alternatively, a user can employ the selection tool 336 to select one or more spatial regions of interest on a cardiac envelope in response to a user input, and the reconstruction engine 330 can adapt the MFS to reconstruct signals on the selected region of interest of the cardiac envelope.

An output generator 338 can generate corresponding output data 324. As described herein the output generator 338 of the mapping system 312 can provide the output data 324 based on the near-field components of electrophysiological signals determined for at least a portion of (e.g., up to including all of) the cardiac envelope. The output data can also include instructions programmed to render the output data 324 as a corresponding graphical output (e.g., a map) 344 in a display 342. For example, the output generator 338 provides the output data 324 to a graphics pipeline of a computing device that supplies the graphical map via an interface to an output device, such as a display 342. The display 342 can include a screen, wearable augmented reality glasses, a heads up display or the like configured to display a graphical visualization, such as including a map 344, generated based on the reconstructed electrophysiological signals that are produced. The graphical output 344 further may include electrophysiological signals (e.g., voltage potentials) reconstructed on the cardiac envelope or a representation of signal features derived from such reconstructed electrophysiological signals. For example, the electrophysiological signals can represent near-field electrophysiological signals, which can be displayed as a graphical map 344 on graphical representation of patient anatomy (e.g., superimposed on a cardiac surface) for one or more time intervals.

Additionally, in some examples, the output data 324 can be utilized by the system 308 in connection with controlling delivery of therapy and/or monitoring electrical characteristics. The control 310 that is implemented can be fully automated control, semi-automated control (partially automated and responsive to a user input) or manual control based on the output data 324 (e.g., including the near-field components of electrophysiological signals). In some examples, the control 310 of the therapy system 306 is configured to utilize the output data 324 to control one or more parameters, which are used the device 306 to deliver a corresponding therapy. In other examples, an individual can view the map 344 generated on the display 342 to manually control the therapy system at a location determined based on this disclosure. Other types of therapy and devices can also be controlled based on the output data 324 and corresponding graphical map 344.

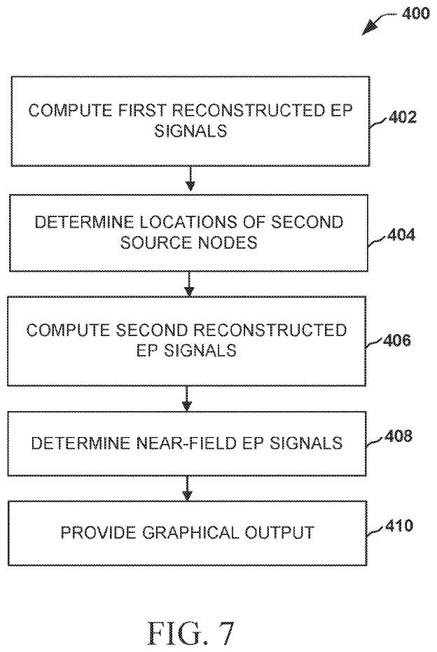

In view of the foregoing structural and functional features described above, FIG. 7 shows an example method 400 that can be performed (e.g., by systems of FIGS. 1, 5 and/or 6) to determine near-field electrophysiological data. Accordingly, reference can be made back to FIGS. 1, 5 and 6 for examples of hardware and software that can be configured to implement the method 400. Different combinations of hardware and software can be used to implement the method 400 in other examples. While, for purposes of simplicity of explanation, the method 400 of FIG. 7 is shown and described as executing serially, it is to be understood and appreciated that the present disclosure is not limited by the illustrated order, as parts of the method could in different orders and/or concurrently from that shown and described herein. Also, the method 400 can be executed by various components configured as machine-readable instructions stored in memory (e.g., one or more non-transitory media) and executable by one or more processors, for example. Moreover, not all illustrated features may be required to implement the method.

At 402, the method 400 includes computing (e.g., by reconstruction engine 112, 202 or 330) first reconstructed electrophysiological signals on a cardiac envelope based on geometry data and electrophysiological data. As described herein, the electrophysiological data represents electrophysiological signals measured (e.g., non-invasively) from locations distributed across a body surface. In some examples, the electrophysiological data can also include invasively measured electrophysiological signals. The geometry data represents geometry for the cardiac envelope and geometry for the locations distributed on the body surface where the electrophysiological signals are measured, such as representative of points and surfaces in a 3D spatial coordinate system. For example, the computations at 402 can be implemented (e.g., by reconstruction engine programmed to perform MFS) using source nodes at respective first source node locations in the 3D spatial coordinate system. As described herein, the source nodes can include body source nodes located outside the body (e.g., radially outward from the body surface), and cardiac source nodes located within the body (e.g., radially inward from the cardiac envelope).

At 404, the first source node locations used at 402 can be located (e.g., by source node placement function 118, 210) to second source node locations to increase the impact of far-field signal components during a second EP reconstruction on the cardiac envelope. At 406, the method 400 includes computing (e.g., by reconstruction engine 112, 202 or 330) second reconstructed electrophysiological signals on the cardiac envelope based on the geometry data and the electrophysiological data. The second EP reconstruction at 406 uses locations for respective source nodes, including the source node locations determined at 404. For example, the second source node locations include body source nodes (e.g., located radially outwardly from the body surface farther than the body source nodes used at 402) and cardiac source nodes (e.g., located radially inward from the cardiac envelope the same or further than the cardiac source nodes used at 402).

At 408, the method includes determining near-field signal components of the electrophysiological signals on at least a portion of the cardiac envelope based on a difference between the first and second reconstructed electrophysiological signals. For example, the determination at 406 can be computed by subtracting the second reconstructed electrophysiological signals from the first reconstructed electrophysiological signals for the respective nodes on the cardiac envelope. At 410, a graphical output can be provided (e.g., on display 432) based on the near-field signal components of the electrophysiological signals determined at 408. In some examples, the method 400 can further use the near-field signal components to control delivery of a therapy, such as by setting one or more therapy parameters used by a therapy device (e.g., device 306) to achieve a desired therapeutic (or subtherapeutic) effect based on the the near-field signal components.

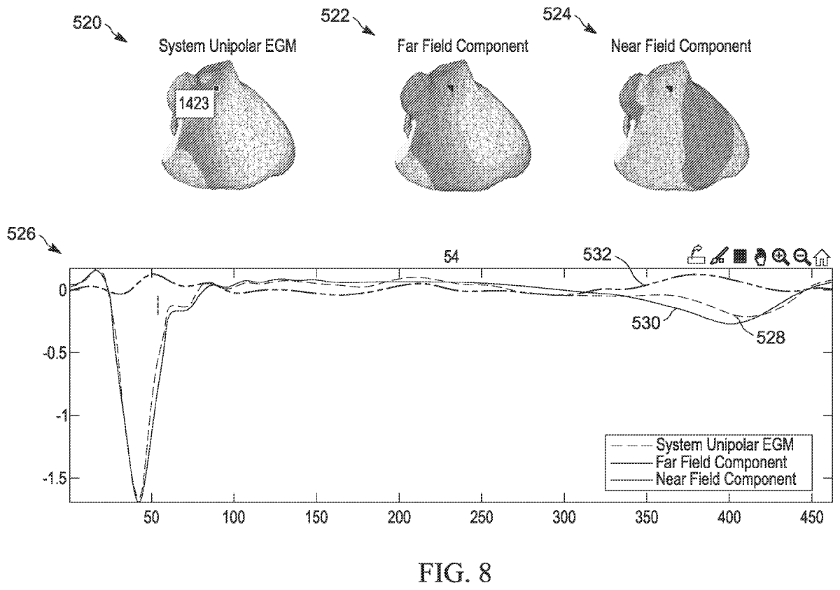

FIGS. 8-17 show different examples of graphical outputs and associated signals, such as can be generated by the system 300 of FIG. 6 or the method 400 of FIG. 7. In each of these examples, near-field electrocardiographic image (ECGI) maps were generated and demonstrated improved accuracy of activation timing over a range of arrhythmia conditions. The maps and EP signals shown in FIGS. 8-17 can be used (e.g., by system 100, 300 and/or method 400) to control one or more parameters of a device that is configured to deliver a therapy (e.g., to achieve a desired therapeutic or subtherapeutic effect), such as described herein.

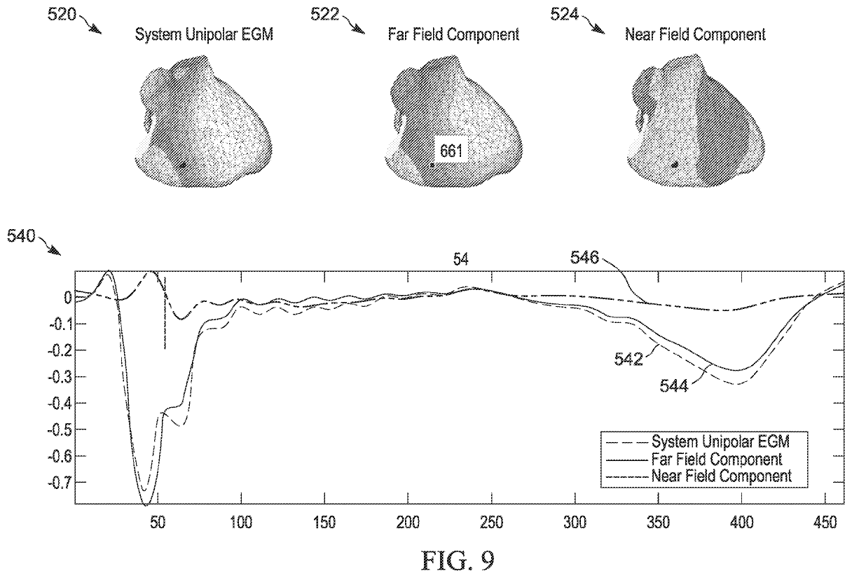

For example, FIGS. 8 and 9 show examples of EP signals and ventricular sinus rhythm maps. FIGS. 8 and 9 show a system ECGI map 520 of EP signals reconstructed on a cardiac envelope (e.g., an epicardial surface), a graphical map 522 of far-field components for EP signals reconstructed on the cardiac envelope, and a graphical map 524 of near-field components for EP signals reconstructed on the cardiac envelope. The system map 520 is generated by a reconstruction engine, such as using nominal source node locations (e.g., as part of MFS), and the far-field map 522 is generated using source node locations adjusted to emphasize far-field signal components. As described herein, the near field map 524 can be derived by subtracting the far-field map from the graphical map 522. FIG. 8 also shows a graph 526 that includes reconstructed EP signals 528, 530 and 532 at a respective location (shown as node 1423 on map 520) on the cardiac envelope for each of the maps 520, 522 and 524. The signal 528 is representative of a system signal for a given cardiac node from the map 520, the signal 530 is representative of a far-field signal for the given cardiac node from the map 522, and the signal 532 is representative of a resulting near-field signal for the given cardiac node from the map 524.

FIG. 9 shows a graph 540 that includes reconstructed EP signals 542, 544 and 546 at another respective location (shown as node 661 on map 520) on the cardiac envelope for each of the maps 520, 522 and 524. The signal 542 is representative of a system signal for a given cardiac node from the map 520, the signal 544 is representative of a far-field signal for the given cardiac node from the map 522, and the signal 546 is representative of a resulting near-field signal for the given cardiac node from the map 524.

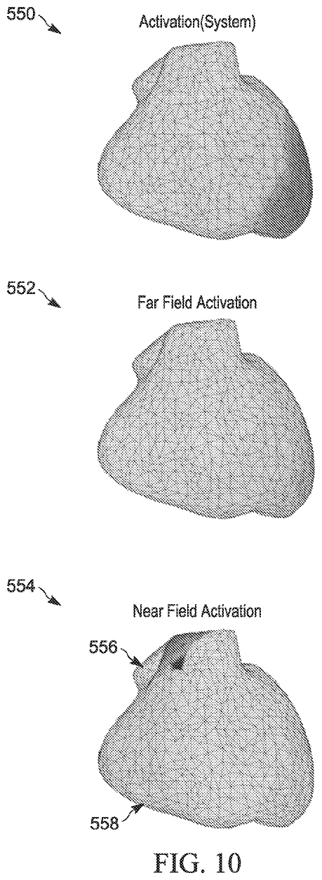

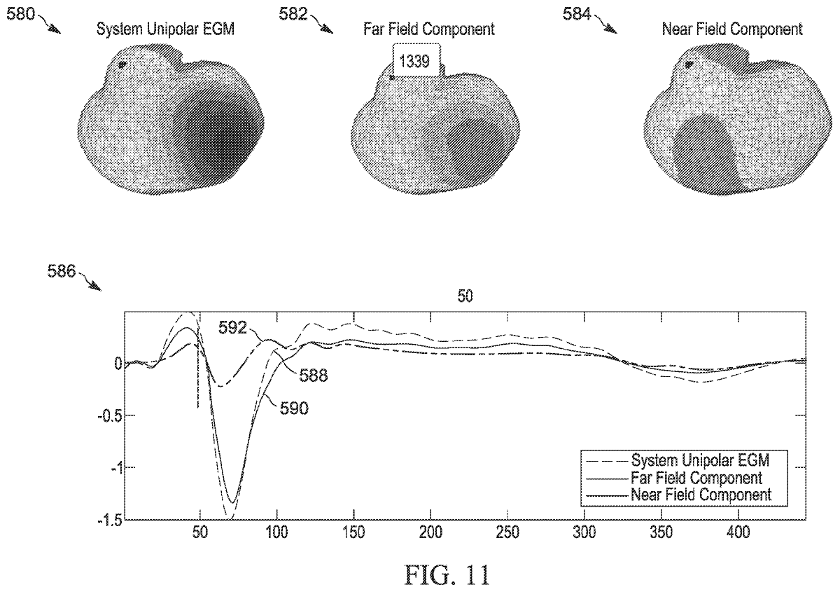

FIG. 10 depicts examples of respective activation maps 550, 552 and 554 derived from reconstructed EP signals on a cardiac envelope based on EP data and geometry data. The activation map 550 shows activation for the system reconstructed EP signals (e.g., using nominal source node locations), and the far-field map 552 is generated from reconstructed EP signals using source node locations that are adjusted to emphasize (increase contribution of) far field signal components. The near-field activation map 554 is derived by determining activation times for EP signals determined across the cardiac enveloped based on a difference between the system electrograms and far-field reconstructed electrograms. Also, the near-field map 554 shows improved near-field activation at regions 556 and 558, in which the effects of far-field signal have been reduced.

As another example, FIGS. 11-13 show examples of EP signals and simus rhythm maps from a patient with supraventricular tachycardia (SVT). FIG. 11 shows a system ECGI map 580 of EP signals reconstructed on a cardiac envelope (e.g., an epicardial surface), an ECGI map 582 of far-field components for EP signals reconstructed on the cardiac envelope, and an ECGI map 584 of near-field components for EP signals reconstructed on the cardiac envelope. The system map 580 is generated by a reconstruction engine, such as using nominal source node locations (e.g., as part of MFS), and the far-field map 582 is generated using source node locations adjusted to emphasize far-field signal components. As described herein, the near field map 584 can be derived by subtracting the far-field map from the graphical map 522.

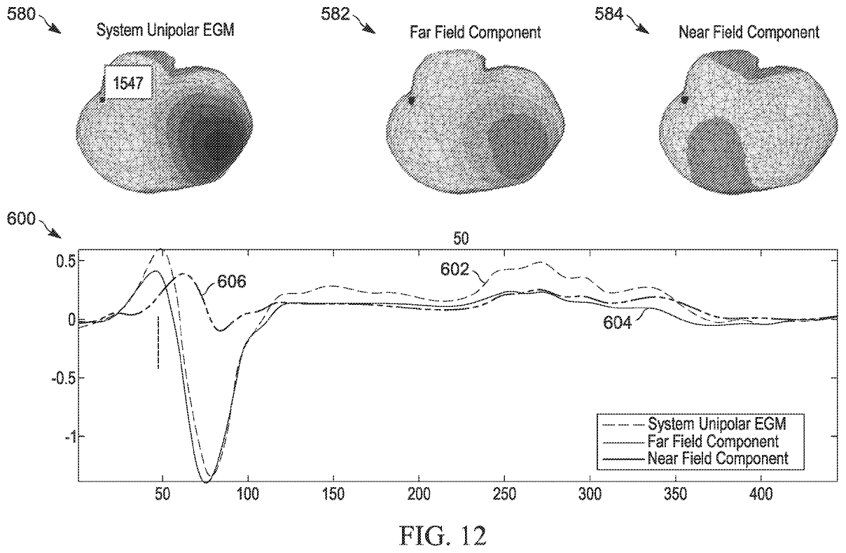

FIG. 11 also shows a graph 586 that includes reconstructed EP signals 588, 590 and 592 at a respective location (shown as node 1339 on map 582) on the cardiac envelope for each of the maps 580, 582 and 584. The signal 588 is representative of a system signal for a given cardiac node from the map 580, the signal 590 is representative of a far-field signal for the given cardiac node from the map 582, and the signal 592 is representative of a resulting near-field signal for the given cardiac node from the map 584. FIG. 12 shows a graph 600 that includes reconstructed EP signals 602, 604 and 606 at another respective location (shown as node 1547 on map 580) on the cardiac envelope for each of the maps 520, 522 and 524. The signal 602 is representative of a system signal for a given cardiac node from the map 580, the signal 604 is representative of a far-field signal for the given cardiac node from the map 582, and the signal 606 is representative of a resulting near-field signal for the given cardiac node from the map 584.

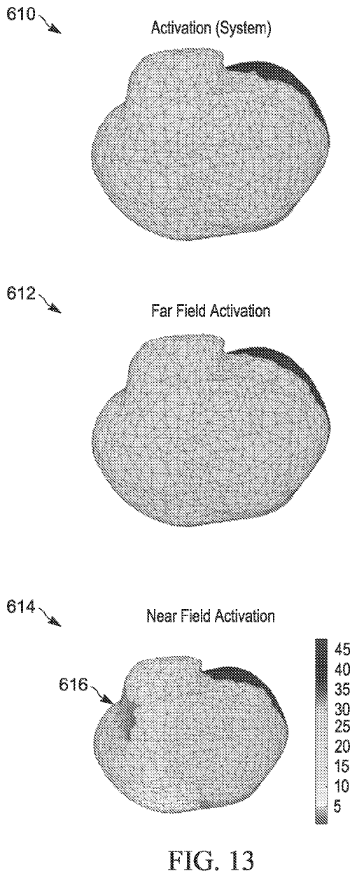

FIG. 13 depicts examples of respective activation maps 610, 612 and 614 derived from reconstructed EP signals on a cardiac enveloped based on EP data and geometry data. The activation map 610 shows activation for the system reconstructed EP signals (e.g., using nominal source node locations), and the far-field map 612 is generated from reconstructed EP signals using source node locations that are adjusted to emphasize (increase contribution of) far field signal components. The near-field activation map 614 is derived by determining activation times for EP signals determined across the cardiac enveloped based on a difference between the system electrograms and far-field reconstructed electrograms. Also, the near-field map 614 shows improved near-field activation at region 616, in which the effects of far-field signal have been reduced.

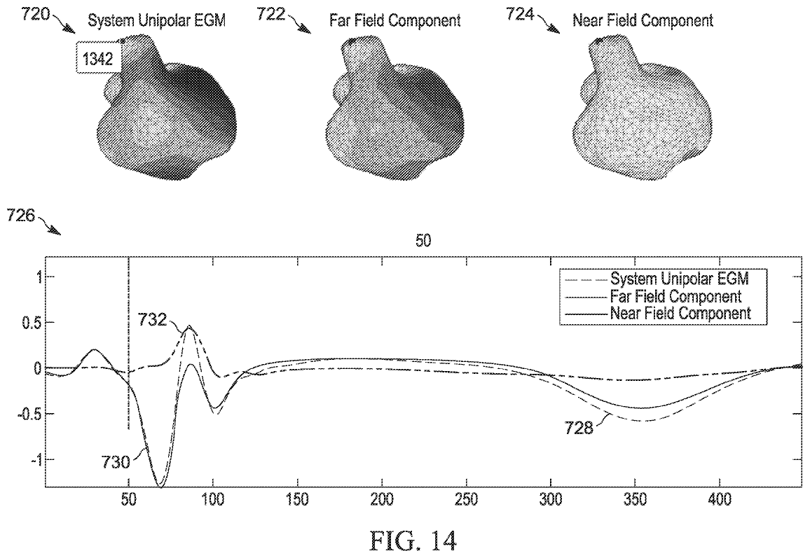

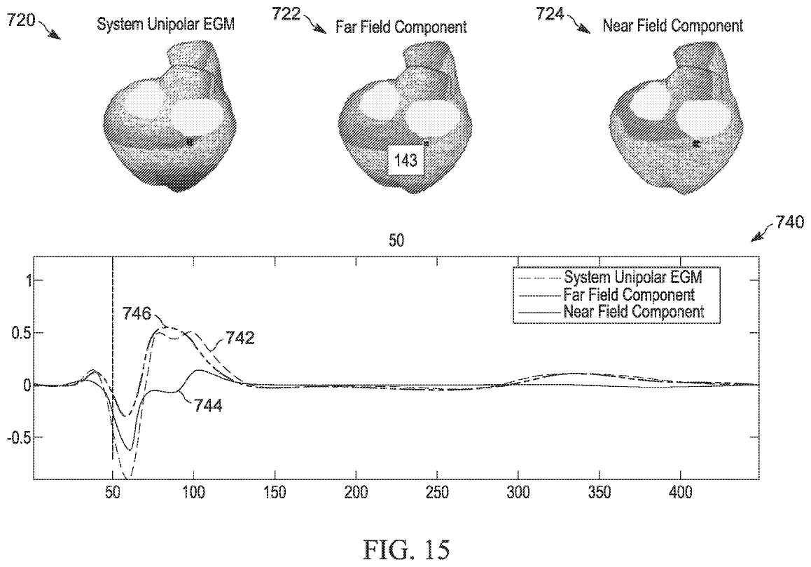

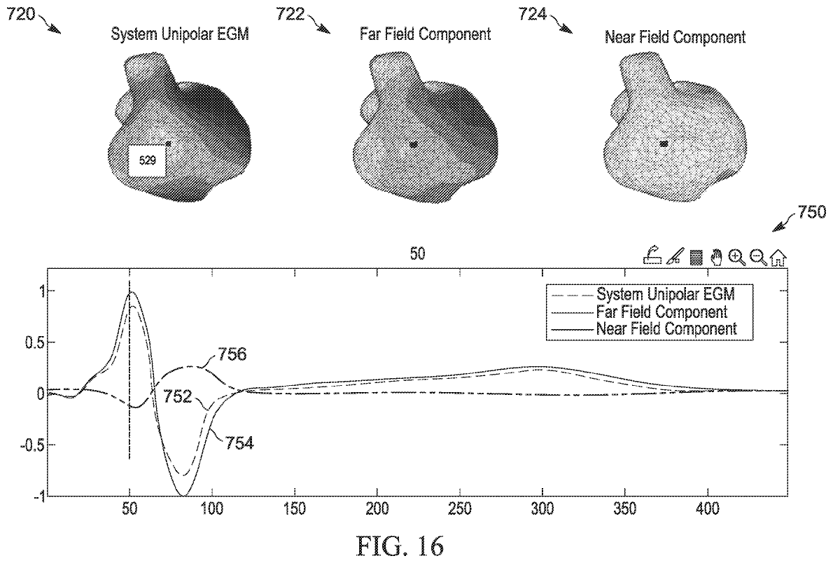

As a further example, FIGS. 14-17 show examples of EP signals and sinus rhythm ventricular maps from patient with a premature ventricular contraction (PVC). FIGS. 14, 15 and 16 show a system ECGI map 720 of EP signals reconstructed on a cardiac envelope (e.g., an epicardial surface), a graphical map 722 of far-field components for EP signals reconstructed on the cardiac envelope, and a graphical map 724 of near-field components for EP signals reconstructed on the cardiac envelope. In FIGS. 14 and 16 the maps are shown at the same viewing angle of the cardiac envelope (e.g., the heart), and a different viewing angle is shown in FIG. 15. The system map 720 is generated by a reconstruction engine, such as using nominal source node locations (e.g., as part of MFS), and the far-field map 722 is generated using source node locations adjusted to emphasize far-field signal components. As described herein, the near field map 724 can be derived by subtracting the far-field map from the graphical map 722.

FIG. 14 also shows a graph 726 that includes reconstructed EP signals 728, 730 and 732 at a respective location (shown as node 1342 on map 720) on the cardiac envelope for each of the maps 720, 722 and 724. The signal 728 is representative of a system signal for a given cardiac node from the map 520, the signal 730 is representative of a far-field signal for the given cardiac node from the map 722, and the signal 732 is representative of a resulting near-field signal for the given cardiac node from the map 724. FIG. 15 shows a graph 740 that includes reconstructed EP signals 742, 744 and 746 at another location (shown as node 143 on map 722) on the cardiac envelope for the respective maps 720, 722 and 724. The signal 742 is representative of a system signal for a given cardiac node from the map 720, the signal 744 is representative of a far-field signal for the given cardiac node from the map 722, and the signal 746 is representative of a resulting near-field signal for the given cardiac node from the map 724. FIG. 16 shows a graph 750 that includes reconstructed EP signals 752, 754 and 756 at another location (shown as node 529 on map 720) on the cardiac envelope for the respective maps 720, 722 and 724. The signal 752 is representative of a system signal at a given cardiac node 529 from the map 750, the signal 754 is representative of a far-field signal for the given node from the map 722, and the signal 756 is representative of a resulting near-field signal for the given cardiac node from the map 724.

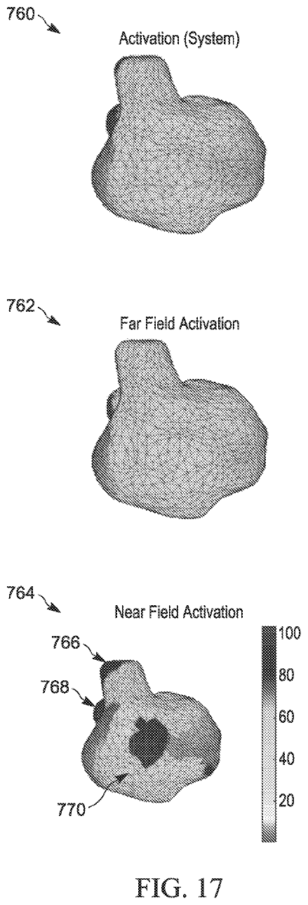

FIG. 17 depicts examples of respective activation maps 760, 762 and 764 derived from reconstructed EP signals on a cardiac enveloped based on EP data and geometry data. The activation map 760 shows activation for the system reconstructed EP signals (e.g., using nominal source node locations), and the far-field map 762 is generated from reconstructed EP signals using source node locations that are adjusted to emphasize (increase contribution of) far field signal components. The near-field activation map 764 is derived by determining activation times for EP signals determined across the cardiac enveloped based on a difference between the system electrograms and far-field reconstructed electrograms. Also, the near-field map 764 shows improved near-field activation at regions 766, 768 and 770, in which the effects of far-field signal have been reduced, as demonstrated by the near-field component signals 732, 746 and 756, respectively.

According to one example, a computer-implemented method includes computing first reconstructed electrophysiological signals on a cardiac envelope based on geometry data and electrophysiological data, wherein the electrophysiological data represents electrophysiological signals measured non-invasively from locations distributed across a body surface, and the geometry data represents geometry for the cardiac envelope and geometry for the locations distributed on the body surface where the electrophysiological signals are measured. The method also includes computing second reconstructed electrophysiological signals on the cardiac envelope based on the geometry data and the electrophysiological data, the second reconstructed electrophysiological signals being representative of far-field signal components. The method also includes determining near-field components of the electrophysiological signals on at least a portion of the cardiac envelope based on the first and second reconstructed electrophysiological signals.

In some implementations, the computing of the first and second reconstructed electrophysiological signals on the cardiac envelope includes using a method of fundamental solutions.

In certain implementations, the using of the method of fundamental solutions includes placing at least some source nodes farther from the cardiac envelope for computing the second reconstructed electrophysiological signals than respective source nodes used to compute the first reconstructed electrophysiological signals.

In some implementations, the placing of at least some source nodes includes placing body source nodes a first uniform distance radially outwardly from the body surface.

In some implementations, the placing of at least some source nodes includes computing a distance between the locations on the body surface where measurements are made and cardiac nodes on the cardiac envelope, and adaptively placing body source nodes radially outwardly from the body surface based on the computed distance.

In certain implementations, the placing of at least some source nodes further includes placing cardiac source nodes a second uniform distance radially inwardly from the cardiac envelope.

In certain implementations, the placing of at least some source nodes includes: computing a distance between the locations on the body surface where measurements are made and respective cardiac nodes on the cardiac envelope; and adaptively placing cardiac source nodes radially inwardly from the cardiac envelope based on the computed distance.

In some implementations, the computing of the second reconstructed electrophysiological signals includes computing an average value of the first reconstructed electrophysiological signals in a spatial neighborhood respective cardiac nodes on the cardiac envelope.

In some implementations, the near-field components of the electrophysiological signals are determined based on a difference between the first and second reconstructed electrophysiological signals.

In some implementations, the electrophysiological signals measured non-invasively from locations distributed across the body surface include unipolar signals.

In some implementations, the method further includes providing a graphical representation based on the near-field components of the electrophysiological signals.

In some implementations, one or more non-transitory computer-readable media have instructions which, when executed by a processor, perform any of the methods, individually or in any combination.

According to another example, a system includes memory to store data and executable instructions, the data including electrophysiological data representing electrophysiological signals measured from locations distributed across a body surface, and geometry data representing geometry for a surface of interest and geometry for the locations distributed on the body surface where the electrophysiological signals are measured. At least one processor is configured to access the memory and execute the instructions to at least: compute first reconstructed electrophysiological signals on the surface of interest based on the geometry data and the electrophysiological data; compute second reconstructed electrophysiological signals on the surface of interest based on the geometry data and the electrophysiological data, the second reconstructed electrophysiological signals being representative of far-field signal components; and determine near-field components for the electrophysiological signals for at least a portion of the surface of interest based on the first and second reconstructed electrophysiological signals for the portion of the surface of interest.

In some implementations, the system further includes an arrangement of electrodes configured to measure the electrophysiological signals from the locations distributed across the body surface, and a display configured to display a graphical visualization generated based on at least one of the reconstructed electrophysiological signals.

In some implementations, the surface of interest includes a cardiac envelope, and the processor is configured to compute each of the first and second reconstructed electrophysiological signals on the cardiac envelope using a method of fundamental solutions.

In some implementations, the method of fundamental solutions includes instructions to: derive an analytical expression for the method of fundamental solutions that includes a matrix A that relates a location of each source node to the locations distributed across the body surface where the electrophysiological signals are measured; perform an inverse computation on the A matrix and the measured electrophysiological signals to compute a plurality of source node coefficients; determine a matrix of coefficients B that relates each cardiac node location on the cardiac envelope to respective source node locations; and perform a forward computation using B and the plurality of source node coefficients to compute the first reconstructed electrophysiological signals on the cardiac envelope.

In certain implementations, the method of fundamental solutions includes node placement instructions to place at least some source nodes farther from the cardiac envelope for computing the second reconstructed electrophysiological signals than respective source nodes used to compute the first reconstructed electrophysiological signals.

In some implementations, the node placement instructions are programmed to place body source nodes a uniform distance radially outwardly from the body surface.

In some implementations, the system the node placement instructions are programmed to: compute a distance between the locations on the body surface where measurements are made and cardiac nodes on the cardiac envelope; and adaptively place body source nodes radially outwardly from the body surface based on the computed distance.

In some implementations, the node placement instructions are further programmed to place cardiac source nodes a second uniform distance radially inwardly from the cardiac envelope.

In some implementations, the node placement instructions are further programmed to: computing a distance between the locations on the body surface where measurements are made and respective cardiac nodes on the cardiac envelope; and adaptively placing cardiac source nodes radially inwardly from the cardiac envelope based on the computed distance.

In some implementations, the instructions are further programmed to compute the second reconstructed electrophysiological signals on the surface of interest are programmed to compute an average value of the first reconstructed electrophysiological signals in a spatial neighborhood respective cardiac nodes on the surface of interest.

In certain implementations, the instructions to compute the near-field components of the electrophysiological signals are programmed to determine the near-field components of the electrophysiological signals based on a difference between the first and second reconstructed electrophysiological signals.

In some implementations, the system, the electrophysiological signals measured from locations distributed across the body surface include unipolar signals.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1. A computer-implemented method comprising:
computing first reconstructed electrophysiological signals on a cardiac envelope based on geometry data and electrophysiological data, wherein the electrophysiological data represents electrophysiological signals measured non-invasively from locations distributed across a body surface, and the geometry data represents geometry for the cardiac envelope and geometry for the locations distributed on the body surface where the electrophysiological signals are measured;
computing second reconstructed electrophysiological signals on the cardiac envelope based on the geometry data and the electrophysiological data, the second reconstructed electrophysiological signals being representative of far-field signal components; and
determining near-field components of the electrophysiological signals on at least a portion of the cardiac envelope based on the first and second reconstructed electrophysiological signals.

2. The method of claim 1, wherein computing the first and second reconstructed electrophysiological signals on the cardiac envelope comprises using a method of fundamental solutions, and
wherein using the method of fundamental solutions comprises placing at least some source nodes farther from the cardiac envelope for computing the second reconstructed electrophysiological signals than respective source nodes used to compute the first reconstructed electrophysiological signals.

3. The method of claim 2, wherein placing at least some source nodes comprises:
placing body source nodes a first uniform distance radially outwardly from the body surface; or
computing a distance between the locations on the body surface where measurements are made and cardiac nodes on the cardiac envelope; and
adaptively placing body source nodes radially outwardly from the body surface based on the computed distance.

4. The method of claim 3, wherein placing at least some source nodes further comprises placing cardiac source nodes a second uniform distance radially inwardly from the cardiac envelope.

5. The method of claim 3, wherein placing at least some source nodes comprises:
computing a distance between the locations on the body surface where measurements are made and respective cardiac nodes on the cardiac envelope; and
adaptively placing cardiac source nodes radially inwardly from the cardiac envelope based on the computed distance.

6. The method of claim 1, wherein computing the second reconstructed electrophysiological signals comprises computing an average value of the first reconstructed electrophysiological signals in a spatial neighborhood respective cardiac nodes on the cardiac envelope.

7. The method of claim 1, wherein the near-field components of the electrophysiological signals are determined based on a difference between the first and second reconstructed electrophysiological signals.

8. The method of claim 1, wherein the electrophysiological signals measured non-invasively from locations distributed across the body surface comprise unipolar signals.

9. The method of claim 1, further comprising providing a graphical representation based on the near-field components of the electrophysiological signals.

10. One or more non-transitory computer-readable media having instructions which, when executed by a processor, cause the processor to perform a method comprising:
computing first reconstructed electrophysiological signals on a cardiac envelope based on geometry data and the electrophysiological data, wherein the electrophysiological data represents electrophysiological signals measured non-invasively from locations distributed across a body surface, and the geometry data represents geometry for the cardiac envelope and geometry for the locations distributed on the body surface where the electrophysiological signals are measured;
computing second reconstructed electrophysiological signals on the cardiac envelope based on the geometry data and the electrophysiological data, the second reconstructed electrophysiological signals being representative of far-field signal components; and
determine near-field components for the electrophysiological signals for at least a portion of the cardiac envelope based on the first and second reconstructed electrophysiological signals.

11. The media of claim 10, wherein computing the first and second reconstructed electrophysiological signals on the cardiac envelope comprises using a method of fundamental solutions, and
wherein using the method of fundamental solutions comprises placing at least some source nodes farther from the cardiac envelope for computing the second reconstructed electrophysiological signals than respective source nodes used to compute the first reconstructed electrophysiological signals.

12. The system of claim 11, wherein placing at least some source nodes comprises:
computing a distance between the locations on the body surface where measurements are made and cardiac nodes on the cardiac envelope; and
adaptively placing body source nodes radially outwardly from the body surface based on the computed distance.

13. The method of claim 12, wherein placing at least some source nodes further comprises placing a cardiac source nodes a second uniform distance radially inwardly from the cardiac envelope.

14. The method of claim 14, wherein placing at least some source nodes comprises:
placing body source nodes a uniform distance radially outwardly from the body surface; or
computing a distance between the locations on the body surface where measurements are made and respective cardiac nodes on the cardiac envelope; and
adaptively placing cardiac source nodes radially inwardly from the cardiac envelope based on the computed distance.

15. A system comprising:
memory to store data and executable instructions, the data including electrophysiological data representing electrophysiological signals measured from locations distributed across a body surface, and geometry data representing geometry for a surface of interest and geometry for the locations distributed on the body surface where the electrophysiological signals are measured; and
at least one processor to access the memory and execute the instructions to at least:
compute first reconstructed electrophysiological signals on the surface of interest based on the geometry data and the electrophysiological data;
compute second reconstructed electrophysiological signals on the surface of interest based on the geometry data and the electrophysiological data, the second reconstructed electrophysiological signals being representative of far-field signal components; and
determine near-field components for the electrophysiological signals for at least a portion of the surface of interest based on the first and second reconstructed electrophysiological signals for the portion of the surface of interest.

16. The system of claim 15, wherein the instructions to compute the near-field components of the electrophysiological signals are programmed to determine the near-field components of the electrophysiological signals based on a difference between the first and second reconstructed electrophysiological signals, and the system further comprises:
an arrangement of electrodes configured to measure the electrophysiological signals from the locations distributed across the body surface; and
a display configured to display a graphical visualization generated based on at least one of the reconstructed electrophysiological signals.

17. The system of claim 16, wherein the surface of interest comprises a cardiac envelope, and
wherein processor is configured to compute each of the first and second reconstructed electrophysiological signals on the cardiac envelope using a method of fundamental solutions, wherein the method of fundamental solutions comprises instructions to:
derive an analytical expression for the method of fundamental solutions that includes a matrix A that relates a location of each source node to the locations distributed across the body surface where the electrophysiological signals are measured;
perform an inverse computation on the A matrix and the measured electrophysiological signals to compute a plurality of source node coefficients;
determine a matrix of coefficients B that relates each cardiac node location on the cardiac envelope to respective source node locations; and
perform a forward computation using B and the plurality of source node coefficients to compute the first reconstructed electrophysiological signals on the cardiac envelope.

18. The system of claim 15, wherein the method of fundamental solutions comprises node placement instructions to place at least some source nodes farther from the cardiac envelope for computing the second reconstructed electrophysiological signals than respective source nodes used to compute the first reconstructed electrophysiological signals, wherein the node placement instructions are programmed to at least one of:
place body source nodes a uniform distance radially outwardly from the body surface; or
compute a distance between the locations on the body surface where measurements are made and cardiac nodes on the cardiac envelope; and
adaptively place body source nodes radially outwardly from the body surface based on the computed distance.

19. The system of claim 18, wherein the node placement instructions are further programmed to:
place cardiac source nodes a second uniform distance radially inwardly from the cardiac envelope; or
compute a distance between the locations on the body surface where measurements are made and respective cardiac nodes on the cardiac envelope; and
adaptively place cardiac source nodes radially inwardly from the cardiac envelope based on the computed distance.

20. The system of claim 15, wherein the instructions to compute the near-field components of the electrophysiological signals are programmed to determine the near-field components of the electrophysiological signals based on a difference between the first and second reconstructed electrophysiological signals.
